(19) United States
(12) Patent Application Publication
(10) Pub. No.: US 2017/0006896 A1
(43) Pub. Date: Jan. 12, 2017

(54) A Method for Improving Maize Digestibility in Bovine Animals

(71) Applicants: DSM IP ASSETS B.V., Heerlen (NL); NOVOZYMES A/S, Bagsvaerd (DK)

(72) Inventors: Antje Schwemer, Berlin (DE); Anna Stenssons, Oberstdorf (Schoellang) (DE); Wolfgang Schlifka, Basel (CH)

(73) Assignees: DSM IP Assets B.V., Heerlen (NL); Novozymes A/S, Bagsvaerd (DK)

(21) Appl. No.: 15/120,550

(22) PCT Filed: Feb. 25, 2015
(86) PCT No.: PCT/EP2015/053908

§ 371 Date: Aug. 22, 2016

(30) Foreign Application Priority Data
Feb. 25, 2014 (EP) 14156583.8

(51) Int. Cl. A23K 20/189; A61K 38/48; A23K 50/10; A61K 38/47; A23K 10/14; A23K 10/30

(52) U.S. Cl. CPC A23K 20/189; A23K 10/14; A23K 10/30; A23K 50/10; A61K 38/47; A61K 38/48

(57) ABSTRACT

The invention relates to the use of at least one bacterial amylase in combination with one or more protease(s) in feed for ruminant animals of the subfamily Bovinae for improving digestibility of maize and/or maize silages, in particular for improving milk yield, weight gain, and/or Feed Conversion Ratio (FCR). Examples of bovine animals are dairy cows and beef cattle. The invention also relates to the use of such enzyme combinations in feed additive compositions and animal feed compositions enriched with maize and maize silages.

### FIELD OF THE INVENTION

High-yielding cows in modern farming systems live under conditions that are characterised by a very high milk production (dairy cows) or growth rate (beef cattle), which is followed by an equally high energy requirement. The utilisation of the feed decreases markedly when intake is increased beyond maintenance levels. In part to account for this, more and more easily degradable feed is included into the ruminant feed e.g. starch-containing raw materials such a cereal-based concentrates and whole cereal silages. The starchy material is frequently recovered in the faeces implying that the utilisation of such feed ingredients could be enhanced further.

Maize or maize silage is becoming more common in increasing amounts in feed rations, in particular in feed for ruminants, due to its efficient growth and for its energy dense properties.

The energy content of feed for bovine animals can be measured using an in-vitro fermentation technique developed at the Institute of Animal Nutrition, University of Hohenheim by Prof. Menke and his colleagues in 1979. The Hohenheim feeding value test (HFT) involves the measurement of the volume of gas produced during a 24 hour incubation of animal feed in rumen fluid. The amount of gas produced during the incubation directly correlates with digestibility of the feed and therefore to the energy content. Since the first publication of this method there has been numerous improvements and adaptations made as described by Steingass and Menke, 1986.

Modifications to the HFT allow the changes in the rate of gas production to be observed. Changing certain aspects of the test such as substrate type, substrate preparation and/or incubation time leads to differences in the available energy of the substrate (feed) to the rumen fluid mixture. The adding of certain substances to the HFT fermentation can increase or decrease the digestibility of the substrate. There is, therefore, a need for consistency of procedures such as the equipment and solutions that are used, the taking of measurements and the preparation of the substrate.

Maize is a seed made up of a germ surrounded by the endosperm which is protected by an outer covering called the pericarp. The endosperm makes up about 80% by weight of the kernel and contains nutrients, primarily starch and varying levels of storage proteins, for germination. Synonyms used to describe two endosperm types are horny, vitreous and translucent for hard endosperms and floury and opaque for soft endosperms.

Maize is a very diverse crop of grain and is characterized by their varying type and size of endosperm although all kernels have the same basic structure. The varieties that are most common are the flint, dent, flour, sweet and pop varieties and reveal the type of endosperm. Flint kernels contain only hard endosperm. Flour kernels contain only soft endosperm. Dent kernels contain soft endosperm surrounded by hard endosperm. During drying of dent kernels the soft endosperm tends to collapse causing “denting” of the top of kernel giving it its name. These three maize varieties are those primarily used for livestock feed. Sweet kernels have a sugary gene in them which hinders the formation of starch from sugar and is mostly for human consumption as are pop(-corn) kernels. Additional varieties include a wide range of ornamental maize that contains varying pigments.

Although high energy maize is more widely available and beneficial for livestock feed the stomach of the ruminant is not able to fully utilize all of it. The rumens ability to break down the hard, vitreous kernels is limited due to the tightly bound starch granules. Maize is, therefore, a good source of stable starch, or “bypass starch”, which are starch molecules that reach the lower digestive tract undigested by the rumen microorganisms. Here the starch is more energy efficiently broken down into glucose molecules and absorbed by the small intestine. The utilization of starch in the small intestine is an important supply of glucose but is also limited in the amount that can be absorbed. The remaining starch is excreted from the ruminant undigested as waste. Reducing the amount of starch that leaves the animal undigested while maintaining a source of stable starch should be possible. With maize being the current preferred energy dense feed for livestock, it is important to understand and increase the digestibility of maize proteins and starches for a more complete utilization by the ruminant.

How easily the maize kernels are broken down depends on the structure of the endosperm and more specifically the amount and type of storage proteins it contains. The storage proteins of maize include albumins, globulins, prolamins and glutelins where prolamin, the alcohol soluble storage protein, tends to be associated with the indigestibility of starch in maize kernels.

Maize prolamin is known as zein and is an alcohol soluble storage protein that makes up about 50-60% of the total protein in the whole kernel. Zein is made up of α-, β-, δ-, and γ-fractions classified by their genetic sequence homology. Zein is found in both hard and soft endosperms of maize kernels but the α-zeins, which make up about 70% of the total kernel zein, are more abundant in hard endosperm. Zein in hard endosperm encases starch granules forming the Prolamin-Starch-Matrix (PSM) giving the maize kernels a vitreous or “glass-like” appearance. The PSM is very difficult for the rumen to break down hindering starch digestion. Variation in protein content and, therefore, zein content of maize is due to but not limited by harvest time, soil types, and environmental circumstances (e.g. drought, excessive rainfall) all of which are linked to the nitrogen fertility status during the growth period. Maize kernels deficient in nitrogen tend to have softer, flourier endosperms containing as little as half the amount of the normal prolamin content.

As a consequence of this, improvements in the digestibility of maize need to be made to allow full utilization of the energy potential and all the available nutrients.

### DESCRIPTION OF THE RELATED ART

WO 03/068256 A1 describes an amylase feed supplement for improved ruminant nutrition. The amylase used is a fungal amylase produced by Aspergillus oryzae. Tricarico et al, in Animal Science 2005, 81: 365-374, describe the effects of Aspergillus oryzae extract containing alpha-amylase activity on ruminal fermentation and milk production in lactating Holstein cows.

Rojo et al (Animal Feed Science and Technology, 123-124 (2005), 655-665) studied the effects of exogenous amylases from Bacillus licheniformis and Aspergillus niger on ruminal starch digestion and lamb performance.

It is an object of the present invention to provide an improved, concept which may alleviate the problems described above by improving feed utilization, milk yield, Feed conversion ratio and/or weight gain if maize or maize silage is included in the feed for bovine animals.

### SUMMARY OF THE INVENTION

It has now been found surprisingly that the combined use of an amylase with one or more proteolytic enzymes has the advantage of being able to significantly improve digestibility of maize in feed for bovine animals.

In particular, the inventors of the present invention have found that the addition of a protease in addition to an amylase supplement results in a significant boost in the rate of gas production when incubated in-vitro using the Hohenheim Feeding Value Test even when the total enzyme dosage was the same or less.

Furthermore, the inventors have found that that the addition of a protease in addition to an amylase supplement results in a significant increase of the in situ dry matter disappearance of feedstuff (ISDMD) containing maize silage or maize grain.

Therefore, the invention relates to a method for improving digestibility of maize and maize silages. In particular the invention relates to a method for improving digestibility of maize and/or maize silages in feed for animals of the subfamily Bovinae, which methods comprise treating the maize source with an efficient amount of one or more proteolytic enzymes in combination with at least one amylase. More specifically, the invention relates to methods for improving digestibility of maize kernels in feed for animals of the subfamily Bovinae, which methods comprise treating the maize source with an efficient amount of one or more amylase, preferably at least one bacterial amylase, in combination with at least one proteolytic enzyme.

The invention further relates to a method for increasing milk yield of animals of the subfamily Bovinae, the method comprising the step of adding at least one bacterial amylase and one or more proteolytic enzymes to the feed which comprises maize and/or maize silages.

The invention further relates to a method for increasing weight gain of animals of the subfamily Bovinae, the method comprising the step of adding at least one bacterial amylase and one or more proteolytic enzymes to the feed which comprises maize and/or maize silages.

The invention further relates to a method for improving the Feed Conversion Ratio of animals of the subfamily Bovinae, the method comprising the step of adding at least one bacterial amylase and one or more proteolytic enzymes to the feed which comprises maize and/or maize silages.

The present invention also relates to the use of one or more proteolytic enzymes in combination with at least one amylase in feed for animals of the subfamily Bovinae such as dairy cows and beef cattle, for improving digestibility of maize and maize silages in the feed. In an embodiment, the use improves milk yield, weight gain, and/or feed conversion ratio (FCR).

The present invention also relates the use of at least one bacterial amylase and one or more proteolytic enzymes in the preparation of a composition for use in a feed for animals of the subfamily Bovinae.

The invention furthermore relates to feed additive compositions comprising at least one bacterial amylase, together one or more proteolytic enzymes, e.g. protease.

### DETAILED DESCRIPTION OF THE INVENTION

In the present context, an amylase is an enzyme that catalyzes the endo-hydrolysis of starch and other linear and branched oligo- and polysaccharides. In a particular embodiment, the amylase for use according to the invention has alpha-amylase activity, viz. catalyzes the endohydrolysis of 1,4-alpha-glucosidic linkages in oligosaccharides and polysaccharides. Alpha-amylases act, e.g., on starch, glycogen and related polysaccharides and oligosaccharides in a random manner, liberating reducing groups in the alpha-configuration.

The amylase for use according to the invention is stable in the presence of protease. The protease stability may be determined by incubating 0.5 mg purified amylase enzyme protein/ml in a buffer at a desired pH (e.g. pH 3, 4, or 5), for the desired time (e.g. 30, 45, 60, 90, or 120 minutes) in the presence of protease (e.g. pepsin, 70 mg/I), and then raising pH to the desired pH (e.g. pH 4, 5, 6, or 7) and measuring residual activity. The residual amylase activity is preferably at least 20%, preferably at least 30, 40, 50, 60, 70, 80, or at least 90% relative to the control (a non-protease-treated sample).

In a preferred embodiment the amylase of the invention is an alpha-amylase (systematical name: 1,4-alpha-D-glucan glucanohydrolase), preferably a bacterial alpha-amylase. In further embodiments, the amylase of the invention belongs to the EC 3.2.1.-group of amylases, such as EC 3.2.1.1 (alpha-amylase), EC 3.2.1.2 (beta-amylase), EC 3.2.1.3 (glucan 1,4-alpha-glucosidase, amyloglucosidase, or glucoamylase), EC 3.2.1.20 (alpha-glucosidase), EC 3.2.1.60 (glucan 1,4-alpha-maltotetraohydrolase), EC 3.2.1.68 (isoamylase), EC 3.2.1.98 (glucan 1,4-alpha-maltohexosidase), or EC 3.2.1.133 (glucan 1,4-alpha-maltohydrolase).

In a preferred embodiment, the amylase for use according to the invention can be, or is, classified as belonging to the EC 3.2.1.1 group. The EC numbers refer to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmWenzyme/index.html.

Amylase activity may be determined by any suitable assay. Generally, assay-pH and assay-temperature may be adapted to the enzyme in question. Examples of assay-pH-values are pH 2, 3, 4, 5, 6, 7, 8, 9, 10, 11, or 12. Examples of assay-temperatures are 30, 35, 37, 40, 45, 50, 55, 60, 65, 70, 80, 90, or 95° C. Preferred pH values and temperatures are in the physiological range, such as pH values of 3, 4, 5, 6, 7, or 8, and temperatures of 30, 35, 37, or 40° C. The following amylase assay can be used: Substrate: Phadebas tablets (Pharmacia Diagnostics; cross-linked, insoluble, blue-coloured starch polymer, which is mixed with bovine serum albumin and a buffer substance, and manufactured into tablets). Assay Temperature: 37° C. Assay pH: 4.3 (or 7.0, if desired). Reaction time: 20 min. After suspension in water the starch is hydrolyzed by the alpha-amylase, giving soluble blue fragments. The absorbance of the resulting blue solution, measured at 620 nm, is a function of the alpha-amylase activity. One Fungal alpha-Amylase Unit (1 FAU) is the amount of enzyme which breaks down 5.26 g starch per hour at the standard assay conditions. A preferred starch is Merck, Amylum solubile Erg. B. 6, Batch 9947275. A more detailed assay description, APTSMYQI-3207, is available on request from Novozymes NS, Krogshoejvej 36, DK-2880 Bagsvaerd, Denmark.

For a taxonomical classification and identification of bacteria reference is made to Bergey's Manual of Systematic Bacteriology (1986), vol 2, ISBNO-683-0783. In the alternative, the well-known 16SrRNA sequence analysis can be used (see e.g. Johansen et al, Int. J. Syst. Bacteriol, 1999, 49, 1231-1240, in particular the Methods section on p. 1233, 2^{nd} column); or taxonomy experts can be consulted, e.g. from DSMZ or other recognized depositary institutes.

As employed herein the term bacterial designates amylases that are derived from bacteria. The term “derived from” includes enzymes obtainable, or obtained, from wild type bacterial strains, as well as variants thereof. The variants may have at least one substitution, insertion, and/or deletion of at least one amino acid residue. The term variant also includes shufflants, hybrids, chimeric enzymes and consensus enzymes. The variants may have been produced by any method known in the art, such as site-directed mutagenesis, random mutagenesis, consensus derivation processes (EP 897985), and gene shuffling (WO 95/22625, WO 96/00343), etc. For the present purposes an amylase variant qualifies as bacterial when at least one bacterial amylase has been used for its design, derivation or preparation. The term bacterial does not refer to a potential recombinant production host but only to the origin of the amylase encoding gene that is hosted by it.

The amylase for use according to the invention is preferably derived from a strain of Bacillus, such as strains of Bacillus amyloliquefaciens, Bacillus circulans, Bacillus halmapalus, Bacillus licheniformis, Bacillus megaterium, Bacillus sp., Bacillus stearothermophilus, and Bacillus subtilis; preferably from strains of Bacillus amyloliquefaciens, Bacillus halmapalus, Bacillus licheniformis, Bacillus sp., Bacillus subtilis, and Bacillus stearothermophilus.

Non-limiting examples of wildtype amylases for use according to the invention are those derived from Bacillus licheniformis, such as Swissprot entry name AMY_BACLI, primary accession number P06278; Bacillus amyloliquefaciens, such as Swissprot entry name AMY_BACAM, primary accession number P00692; Bacillus megaterium, such as Swissprot entry name AMY_BACME, primary accession number P20845; Bacillus circulans, such as Swissprot entry name AMY_BACCI, primary accession number P08137; Bacillus stearothermophilus, such as Swissprot entry name AMY_BACST, primary accession number P06279. Another example is from Bacillus subtilis, such as Swissprot entry name AMY_BACSU, primary accession number P00691.

For purposes of the present invention, preferred amylases are the amylases contained in the following commercial products: BAN, Stainzyme, Termamyl SC, Natalase, and Duramyl (all from Novozymes), and in the Validase BAA and Validase HT products (from Valley Research). Further particular examples of amylases for use according to the invention are the amylases contained in the following commercial products: Clarase, DexLo, GC 262 SP, G-Zyme G990, G-Zyme G995, G-Zyme G997, G-Zyme G998, HTAA, Optimax 7525, Purastar OxAm, Purastar ST, Spezyme AA, Spezyme Alpha, Spezyme BBA, Spezyme Delta AA, Spezyme DBA, Spezyme Ethyl, Spezyme Fred (GC521), Spezyme HPA, and Ultraphlow (all from Genencor); Validase HT340L, Valley Thin 340L (all from Valley Research); Avizyme 1500, Dextro 300 L, Kleistase, Maltazyme, Maxamyl, Thermozyme, Thermatex, Starzyme HT 120 L, Starzyme Super Conc, and Ultraphlo.

In a particular embodiment, the amylase for use according to the invention is pelleting stable, and/or thermostable. The melting temperature (Tm) of an enzyme is a measure of its thermostability. The amylase of the invention may have a Tm of at least 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C. or at least 95° C., as determined by Differential Scanning calorimetry (DSC). The DSC is performed in a 10 mM sodium phosphate, 50 mM sodium chloride buffer, pH 7.0. The scan rate is constant, e.g. 1.5° C./min. The interval scanned may be from 20 to 100° C. Another buffer may be selected for the scanning, e.g. a buffer of pH 5.0, 5.5, 6.0, or pH 6.5. In further alternative embodiments, a higher or lower scan rate may be used, e.g. a lower one of 1.4° C./min, 1.3° C./min, 1.2° C./min, 1.1° C./min, 1.0° C./min, or 0.9° C./min.

In another preferred embodiment, the amylase for use according to the invention has an activity at pH 7.0 and 37° C. of at least 35% relative to the activity at the pH-optimum and 37° C. More preferably, the activity at pH 7.0 and 37° C. is at least 40, 45, 50, 55, 60, 65, 70, or at least 75% of the activity at the pH-optimum and 37° C.

In another preferred embodiment, the amylase of the invention has an activity at pH 7.0 and 37° C. and in the presence of 5 mM bile salts of at least 25% relative to the activity at the pH-optimum and 37° C. in the absence of bile salts. More preferably, the activity at pH 7.0 and 37° C. and in the presence of 5 mM bile salts is at least 30, 35, 40, 45, 50, 55, 60, or at least 65% of the activity at the pH-optimum and 37° C. in the absence of bile salts. A commercially available bacterial amylase for use according to the present invention is RumiStar® (DSM Nutritional Products AG).

Proteases, or peptidases, catabolize peptide bonds in proteins breaking them down into fragments of amino acid chains, or peptides.

Proteases are classified on the basis of their catalytic mechanism into the following groups: serine proteases (S), cysteine proteases (C), aspartic proteases (A), metalloproteases (M), and unknown, or as yet unclassified, proteases (U), see Handbook of Proteolytic Enzymes, A. J. Barrett, N. D. Rawlings, J. F. Woessner (eds), Academic Press (1998), in particular the general introduction part.

Proteases for use according to the invention are acid stable proteases.

In a particular embodiment, the protease for use according to the invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i. e. the term covers the expression of wild-type, naturally occurring proteases, as well as expression in any host of recombinant, genetically engineered or synthetic proteases.

Examples of microorganisms are bacteria, e. g. bacteria of the phylum Actinobacteria phy. nov., e. g. of class I: Actinobacteria, e. g. of the Subclass V: Actinobacteridae, e. g. of the Order I: Actinomycetales, e. g. of the Suborder XII: Streptosporangineae, e. g. of the Family II: Nocardiopsaceae, e. g. of the Genus I: Nocardiopsis, e. g. Nocardiopsis sp. NRRL 18262, and Nocardiopsis alba; e.g. of the species Bacillus or mutants or variants thereof exhibiting protease activity. This taxonomy is on the basis of Berge's Manual of Systematic Bacteriology, 2nd edition, 2000, Springer (preprint: Road Map to Bergey's).

Preferred proteases according to the invention are acid stable proteases preferably acid stable serine proteases. In a further preferred embodiment, the acid stable serine proteases are 51 serine proteases. Preferred proteases according to the invention are acid stable serine proteases obtained or obtainable from the order Actinomycetales, such as those derived from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases. Other preferred proteases are those described in WO 2001/058276, WO 2004/111220, WO 2004/111221, WO 2004/072221, WO 2005/123911, WO 2013/026796, WO 2013/098185, WO 2013/110766, WO 2013/189972, WO 2014/122161 and WO 2014/037438.

The term serine protease refers to serine peptidases and their clans as defined in the above Handbook. In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. Examples of serine proteases for use according to the invention are proteases of Clan SA, e. g. Family S2 (Streptogrisin), e. g. Sub-family S2A (alpha-lytic protease), as defined in the above Handbook.

Protease activity can be measured using any assay, in which a substrate is employed, that includes peptide bonds relevant for the specificity of the protease in question.

There are no limitations on the origin of the acid stable serine protease for use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by Random Mutagenesis. The preparation of consensus proteins is described in e. g. EP 0 897 985.

Examples of acid-stable proteases for use according to the invention are proteases derived from Nocardiopsis sp. NRRL 18262, and Nocardiopsis alba and proteases of at least 60, 65, 70, 75, 80, 85, 90, or at least 95% amino acid identity to any of these proteases.

For calculating percentage identity, any computer program known in the art can be used. Examples of such computer programs are the Clustal V algorithm (Higgins, D. G., and Sharp, P. M. (1989), Gene (Amsterdam), 73, 237-244; and the GAP program provided in the GCG version 8 program package (Program Manual for the Wisconsin Package, Version 8, Genetics Computer Group, 575 Science Drive, Madison, Wis., USA 53711) (Needleman, S. B. and Wunsch, C. D., (1970), Journal of Molecular Biology, 48, 443-453.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

In another particular embodiment, the protease for use according to the invention, besides being acid-stable, is also thermostable.

The term thermostable means for proteases one or more of the following: That the temperature optimum is at least 50° C., 52° C., 54° C., 56° C., 58° C., 60° C., 62° C., 64° C., 66° C., 68° C., or at least 70° C.

A commercially available serine proteases derived from Nocardiopsis is Ronozyme®ProAct® (DSM Nutritional Products AG).

In the use according to the invention the protease and the amylase can be fed to the animal before, after, or simultaneously with the diet of the animal. The latter is preferred.

In a particular embodiment, the amylase is an alpha-amylase and the protease is a serine protease. In a further particular embodiment, the amylase is a bacterial alpha-amylase and the protease is an acid stable serine protease. In a further particular embodiment, the amylase is a bacterial alpha-amylase and the protease is an acid stable 51 serine protease. In a further particular embodiment, the amylase is a bacterial alpha-amylase and the protease is an acid stable serine protease obtained or obtainable from the order Actinomycetales.

In a particular embodiment, the amylase and the protease, in the form in which they are added to the feed, or when being included in a feed additive, are well-defined. Well-defined means, that the enzyme preparation is at least 50% pure on a protein-basis. In other particular embodiments the enzyme preparation is at least 60, 70, 80, 85, 88, 90, 92, 94, or at least 95% pure. Purity may be determined by any method known in the art, e.g. by SDS-PAGE, or by Size-exclusion chromatography (see Example 12 of WO 01/58275).

A well-defined enzyme preparation is advantageous. For instance, it is much easier to dose correctly to the feed an enzyme that is essentially free from interfering or contaminating other enzymes. The term dose correctly refers in particular to the objective of obtaining consistent and constant results, and the capability of optimising dosage based upon the desired effect.

Enzyme preparations with purities of this order of magnitude are in particular obtainable using recombinant methods of production, whereas they are not so easily obtained and also subject to a much higher batch-to-batch variation when produced by traditional fermentation methods.

The bacterial amylase and protease for use according to the invention are included in bovine diets or bovine feed additives in effective amounts. It is presently contemplated that an effective amount is below 500 mg enzyme protein per kg diet dry matter, preferably below 450, 400, 350, 300, 250, 200, 150, 100, 90, 80, 70, 60, 50, 40, 30, 20, 15, or below 10 mg enzyme protein per kg diet dry matter (ppm) for each enzyme. On the other hand, an effective amount may be above 0.5 mg enzyme protein per kg diet dry matter, preferably above 1, 2, 3, 5, 10, 15, 20 or above 25 mg enzyme protein per kg diet dry matter (ppm). Accordingly, non-limiting examples of preferred dose ranges are: 10-400 mg enzyme protein/kg, preferably 50-300 mg enzyme protein/kg for each enzyme.

In the present context, an animal of the subfamily Bovinae (also called bovines, or bovine animals) means an animal of the kingdom of Animalia, the phylum of Chordata, the class of Mammalia, the order of Artiodactyla, and the family of Bovidae.

This biological subfamily includes about 24 species of medium-sized to large ungulates, including domestic cattle, Bison, the Water Buffalo, the Yak, and the four-horned and spiral-horned antelopes. General characteristics include a cloven-hoof and usually at least one of the sexes of a species having a true horn.

Preferred genera include Tetracerus, Boselaphus, Bubalus, Bos, Pseudoryx, Syncerus, Bison, Tragelaphus, and Taurotragus. A most preferred genera is Bos, which includes the species of Aurochs (Bos primigenius, extinct), Banteng (Bos javanicus), Gaur (Bos frontalis), Yak (Bos mutus), Domestic Cattle (Bos taurus, Bos indicus (today often counted as B. primigenius), and Kouprey (Bos sauveli). For the present purposes, Domestic cattle are the most preferred species. For the present purposes the term includes all races of domestic cattle, and all production kinds of cattle, in particular dairy cows and beef cattle.

Bovines are ruminants, which are characterised by having additional fermentation capacity compared to mono-gastric animals. For example, cows and sheep have three fore-stomachs before the abomasum. The functionally most important is the rumen, which serve as a chamber for feed storage and fermentation. The fermentative processes are carried out by a large and complex flora of anaerobic microorganisms (bacteria, protozoa and fungi). These can degrade cell wall material in addition to protein and starch, thus allowing the ruminant animal to ingest and benefit from feed material that is otherwise not degraded in the abomasum or small intestine. This includes for example hay, other forages and silages rich in cell wall material.

The products of the fermentation in the rumen are short-chain fatty acids (SOFA), which serve as a primary energy source in ruminants, and gasses such as methane and carbon dioxide. In in vitro rumen systems, the volume of gas production is therefore often taken as a measure of the fermentability of a given feed, and an increased gas production in vitro is taken as a measure of improved feed degradation and increased energy availability. Most in vitro systems include the use of freshly sampled rumen fluid, typically from sheep or cows.

Optimal milk production requires sufficient energy intake and thus preferably good feed utilisation by dairy cows. The same is true for obtaining optimal weight gain of beef cattle.

It is contemplated that the combined use of at least one amylase together with one or more protease for use according to the invention significantly improves the degradation in the rumen of maize and maize silages, in particular slowly degradable maize (such as maize kernels), thereby contributing more energy to the rumen microorganisms and to the ruminant itself (in the form of short-chain fatty acids).

It is contemplated, that the improved degradation of maize will give more energy to the cows and thus increase milk yield or weight gain.

For the present purposes, an improved milk yield means either of the following: (i) An increased volume of milk production per day (l/day); (ii) an increased weight of milk production per day (kg/day); (iii) an increased ratio of kg milk produced relative to dry matter intake in kg per day (kg milk/kg DMI); (iv) an increased weight of milk fat produced per day (kg/day); (v) an increased weight of milk protein produced per day (kg/day); (vi) an increased production of 3.5% fat corrected milk per day (kg/day); and/or (vii) an increased production of milk solids per day, wherein the term “milk solids” includes the total amount of lactose, fat, and protein.

An increased milk yield is obtained, e.g., when the dry matter content of the milk increases (e.g. more fat or protein) without a concomitant volume increase, when the volume increases without an increase in the dry matter, and when the volume, as well as the dry matter content of the milk increases.

In particular embodiments herein,
a) the daily milk production (kg/day) is increased by at least 1%, preferably 2, 3, 4, 5, 6, 7, 8, or at least 9%, relative to a control without added amylase and protease;

b) the ratio of daily milk production (kg/day) relative to dry matter intake (DMI) (kg/day) (kg milk/kg DMI) is improved, relative to a control without added amylase and protease, by at least 1%, preferably by at least 2, 3, or at least 4%;
c) the weight of milk fat produced per day (kg/day) is improved, relative to a control without added amylase and protease, by at least 1%, preferably by at least 2, 3, 4, 5, 6, 7, or at least 8%;
d) the weight of milk protein produced per day (kg/day) is improved, relative to a control without added amylase and protease, by at least 1%, preferably by at least 2, 3, 4, 5, 6, 7, 8, or by at least 9%; and/or
e) the production of 3.5% fat corrected milk per day (kg/day) is improved, relative to a control without added amylase and protease, by at least 1%, preferably by at least 2, 3, 4, 5, 6, 7, 8, or by at least 9%.

Embodiments (a)-(e) preferably refer to a cow trial as described in the below FCR-paragraph.

In other particular embodiments herein,
a) the daily milk production (kg/day) is increased by at least 1%, preferably 2, 3, 4, 5, 6, 7, 8, or at least 9%, relative to a control with added amylase but without added protease;
b) the ratio of daily milk production (kg/day) relative to dry matter intake (DMI) (kg/day) (kg milk/kg DMI) is improved, relative to a control with added amylase but without added protease, by at least 1%, preferably by at least 2, 3, or at least 4%;
c) the weight of milk fat produced per day (kg/day) is improved, relative to a control with added amylase but without added protease, by at least 1%, preferably by at least 2, 3, 4, 5, 6, 7, or at least 8%;
d) the weight of milk protein produced per day (kg/day) is improved, relative to a control with added amylase but without added protease, by at least 1%, preferably by at least 2, 3, 4, 5, 6, 7, 8, or by at least 9%; and/or
e) the production of 3.5% fat corrected milk per day (kg/day) is improved, relative to a control with added amylase but without added protease, by at least 1%, preferably by at least 2, 3, 4, 5, 6, 7, 8, or by at least 9%.

Embodiments (a)-(e) preferably refer to a cow trial as described in the below FCR-paragraph.

The Feed Conversion Ratio (FCR) is indicative of how effectively a feed is utilized. The lower the FCR, the better the feed is utilized. The FCR may be determined on the basis of a cow trial comprising a first treatment in which the amylase and protease for use according to the invention are added to the animal feed in a desired concentration (e.g., 6 or 30 mg enzyme protein per kg feed), and a second treatment (control) with no addition of the enzymes to the animal feed, each treatment consisting of four cows, e.g. male or female, the cows being housed in a barn equipped with Calan gates for the measurement of individual feed intake, the cows being fed a TMR diet, the FCR being calculated as the feed intake in kg/cow relative to the weight gain in kg/cow for a desired period of the trial (e.g. the first, the second, the third, or the fourth 21-days periods, or the whole 84-days period), the FCR for the first treatment being improved relative to the FCR of the second treatment. In particular embodiments, the FCR is improved (i.e., reduced) as compared to the control by at least 1.0%, preferably at least 1.5%, 1.6%, 1.7%, 1.8%, 1.9%, 2.0%, 2.1%, 2.2%, 2.3%, 2.4%, or at least 2.5%. In further particular embodiments, the FCR is improved (i.e. reduced) as compared to the control by at least 2.6%, 2.7%, 2.8%, 2.9%, or at least 3.0%. In still further particular embodiments, the FCR is improved (i.e., reduced) as compared to the control by at least 3.1%, 3.2%, 3.3%_{,} 3.4%, 3.5%, 3.6%, 3.7%, or at least 3.8%. In other particular embodiments, the FCR is improved (i.e., reduced) as compared to the control with added amylase but without added protease by at least 1.0%, preferably at least 1.5%, 1.6%, 1.7%, 1.8%, 1.9%, 2.0%, 2.1%, 2.2%, 2.3%, 2.4%, or at least 2.5%. In further particular embodiments, the FCR is improved (i.e. reduced) as compared to the control with added amylase but without added protease by at least 2.6%, 2.7%, 2.8%, 2.9%, or at least 3.0%.

An improved weight gain means an improved daily, weekly, bi-weekly, or monthly weight gain (in g or kg per the relevant time period), relative to a control without added amylase and protease. This is preferably determined in a trial as described in the above FCR-paragraph. In particular embodiments the weight gain is at least 101, 102, 103, 104, 105, 106, 107, 108, 109, or at least 110% of the control (no enzyme addition). In other particular embodiments the weight gain is at least 101, 102, 103, 104, 105, 106, 107, 108, 109, or at least 110% of the control with added amylase but without added protease.

As regards feed compositions for bovines such as cows, as well as ingredients thereof, the bovine diet is usually composed of an easily degradable fraction (named concentrate) and a fibre-rich less readily degradable fraction which in accordance with the present invention comprises as major part maize (corn). Silage is an ensiled version of the fibre-rich fraction, whereby material with a high water content is treated with a controlled anaerobic fermentation process (naturally-fermented or additive treated). Thus the feed composition comprises at least one bacterial amylase, preferably alpha-amylase, together with one or more proteolytic enzymes, preferably acid stable serine protease, and maize and/or maize silage. The bacterial amylase and protease for use according to the invention are included in bovine diets or bovine feed additives in effective amounts.

An effective amount of amylase and proteolytic enzymes in the feed composition is below 500 mg enzyme protein per kg diet dry matter, preferably below 450, 400, 350, 300, 250, 200, 150, 100, 90, 80, 70, 60, 50, 40, 30, 20, 15, or below 10 mg enzyme protein per kg diet dry matter (ppm) for each enzyme. On the other hand, an effective amount may be above 0.5 mg enzyme protein per kg diet dry matter, preferably above 1, 2, 3, 5, 10, 15, 20 or above 25 mg enzyme protein per kg diet dry matter (ppm). Accordingly, non-limiting examples of preferred dose ranges are: 10-400 mg enzyme protein/kg, preferably 50-300 mg enzyme protein/kg for each enzyme.

In a further embodiment, the feed composition improves the weight gain, FCR and/or milk yield of the animals of the subfamily Bovinae as described herein. In an embodiment, the feed composition improves the weight gain of the animals of the subfamily Bovinae by at least 101, 102, 103, 104, 105, 106, 107, 108, 109, or at least 110% of the control (no enzyme addition, or control with added amylase but without added protease). In an embodiment, the feed composition improves the FCR of the animals of the subfamily Bovinae as compared to the control (no enzyme addition, or control with added amylase but without added protease) by at least 1.0%, preferably at least 1.5%, 1.6%, 1.7%, 1.8%, 1.9%, 2.0%, 2.1%, 2.2%, 2.3%, 2.4%, or at least 2.5%. In an embodiment, the feed composition improves the milk yield of the animals of the subfamily Bovinae as described herein.

The feed additive composition of the invention comprises, in addition to the amylase and protease for use according to the invention as described herein above, at least one additional ingredient selected from amongst vitamins and minerals. For example, the feed additive of the invention may include (i) at least one vitamin, (ii) at least one mineral, or (iii) at least one vitamin and at least one mineral.

The at least one vitamin may be fat-soluble or water-soluble. Examples of fat-soluble vitamins are vitamin A, vitamin D3, vitamin E, and vitamin K, e.g. vitamin K3. Examples of water-soluble vitamins are vitamin B12, biotin and choline, vitamin B1, vitamin B2, vitamin B6, niacin, folic acid and panthothenate, e.g. Ca-D-panthothenate.

The at least one mineral may be a macro minerals and/or a trace mineral. Examples of trace minerals are manganese, zinc, iron, copper, iodine, selenium, and cobalt. Examples of macro minerals are calcium, phosphorus and sodium.

Premixes are recognized terms in the art for certain feed additives. They may be solid or liquid. A mineral premix is a composition which is intended for addition to animal feed and which comprises desired kinds and amounts of minerals, in particular trace minerals. A vitamin premix is a composition which is intended for addition to animal feed and which comprises desired kinds and amounts of vitamins. Some premixes include both vitamins and minerals.

In a further embodiment, the feed additive composition comprises at least one bacterial amylase, preferably alpha-amylase, together with one or more proteolytic enzymes, preferably acid stable serine protease, for use in an animal feed composition for animals of the subfamily Bovinae for improving digestibility of maize and maize silages in the feed.

The present invention is further described by the following examples which should not be construed as limiting the scope of the invention.

### EXAMPLES

Chemicals used as buffers and substrates were commercial products of at least reagent grade.

### Example 1

### Modified Hohenheim Forage Value Test (HFT)

The Hohenheim Forage value Test (HFT) is described by Menke et al (1979), J. Agric. Sci. Camb. 93, 217-222: “The estimation of the digestibility and metabolizable energy content of ruminant feedinggstuffs from the gas production when they are incubated with rumen liquor in vitro”, by Steingass, H. (1983): “Bestimmung des energetischen Futterwertes von wirtschaftseigenen Futtermitteln aus der Gasbildung bei der Pansensaftfermentation in vitro” Hohenheim Universität, Fak. Agrarwiss. Dissertation, and by Steingass et al in Tierernahrung 14; pp 251-270 (1986): “Schätzung des energetischen Futterwertes aus der in vitro mit Pansensaft bestimmten Gasbildung and der chemischen Analyse. 1. Untersuchungen zur Methode Übers. Its purpose is primarily to estimate the net energy for lactation in feeds for milk production on the basis of gas production.

A modified version of this test as presented below can be used for testing the effect of exogenous enzymes in a rumen in vitro system.

In brief, the feed substrate is weighed into a glass syringe together with a composition of rumen liquor and an appropriate mixture of buffers. The glass syringe is closed with a close-fitting but movable piston allowing for the increasing volume of the produced gas. The syringe is incubated at 39° C. for 2 to 4 h. The quantity of produced gas is measured and put into a formula for conversion.

Reagents

Mass Element Solution:

6.2 g potassium dihydrogen phosphate (KH2PO4)

0.6 g magnesium sulfate heptahydrate (MgSO4*7H2O)

9 ml concentrated phosphoric acid (1 mol/l)

dissolved in aqua dist. ad 1 l (pH about 1.6)

Buffer Solution:

35.0 g sodium hydrogen carbonate (NaHCO₃)

4.0 g ammonium hydrogen carbonate ((NH₄)HCO₃)

dissolved in aqua dist. ad 1 l

Trace Element Solution:

13.2 g calciumchloride dihydrate (CaCl₂*2H₂O)

10.0 g manganese(II) chloride tetrahydrate (MnCl₂*4H₂O)

1.0 g cobalt(II) chloride hexahydrate (0001₂*6H₂O)

8.0 g iron(III) chloride (FeCl₃*6H₂O)

dissolved in aqua dist. ad 100 ml

Sodium Salt Solution:

100 mg sodium salt

dissolved in aqua dist. ad 100 ml

Reduction Solution:

First 3 ml sodium hydroxide (c=1 mol/l), then 427.5 mg sodium sulphide hydrate (Na₂S*H₂O) is added to 71.25 ml H₂O. The solution is prepared shortly before it is added to the medium solution

Enzyme Buffer:

10.88 g sodium acetate trihydrate (CH₃COONa*3H₂O)

5.88 g calciumchloride-dihydrate (CaCl₂*2H₂O)

0.1 g BSA (bovine serum albumin)

dissolved in aqua dist. ad 2 l, with acetic acid adjusted to pH=5,8

Equipment:
Syringe (glass injection, 100 ml, 1/1 graduated with capillary base)
A silicon tube (for each syringe piece of about 50 mm), which was pulled over the capillary base and can be closed with a clamp
Rotor with power unit for 65 syringes, about 1 rotation per minute
Incubator with ventilator (precision+0.5° C., minimum size inside: 70 cm*70 cm*50 cm)
A precision or analytical balance
Suction pump (e. g. hand-operated, adapted air-pump for motorcycles) to remove the content of rumen, return valve, washing flask
Feeding bottle (2 l) with plug to collect rumen liquor
Gas bottle with technical carbon dioxide and reduction valve
Equipment to fill the rumen liquor, consisting of: a semi-automatic pipette (50 ml), a
Woulff bottle (2 l), a magnetic stirrer, a thermostat with circulation pump and a PVC-bowl (10 l)

### Example 2

### Maize-Degradation In Vitro

1. Materials and Reagent Preparation for HFT

Using an in vitro rumen system as described in Example 1, maize degradation of six different maize samples was determined.

Enzyme Preparations

Two enzyme preparations were supplied by Novozymes, Denmark. The first enzyme is an alpha-amylase marketed for use in dairy cattle as Ronozyme® Rumistar®. Rumistar® has an enzyme activity of 30,860 U/g (In-lab results). The activity was measured using the dinitrosalicylic acid (DNSA) test.

The second enzyme preparation is Ronozyme®ProAct®. Ronozyme®ProAct® is a protease not yet marketed for use in ruminants. The protease has an activity of 75,000 PROT/g and the enzyme preparation contains 44.5 AEP/g.

The enzyme preparations are diluted with distillated water so that 200,000 ppm enzyme preparation is in 200 μL which can be added to the fermentation syringes through the silicon tube. For the enzyme mixture 100 μL of each enzyme dilution is added to the syringe providing 100,000 ppm of each enzyme preparation per fermentation.

Mass Element Solution

The mass element solution is prepared in a one liter measuring flask. The solution contains the following chemicals: 6.2 g potassium dihydrogen phosphate, 0.6 g magnesium sulphate heptahydrogen and, first adding some distilled water, 9.0 mL concentrated phosphoric acid (85%). The phosphoric acid is handled while working under a fume-hood while wearing safety glasses and nitrile gloves. The solution is filled to one liter with distilled water. The mass element solution should have a pH of approximately 1.6. The solution is to be stored at 5-8° C.

Buffer Solution

The Buffer solution is prepared in a one liter measuring flask. The solution contains 35.0 g sodium hydrogen carbonate, 4.0 g ammonium hydrogen carbonate and is filled up to one liter with distilled water. The solution is to be stored at 5-8° C.

Trace Element Solution

The trace element solution is prepared in a 100 ml measuring flask while working under a fume-hood and wearing nitrile gloves. The solution contains 13.2 g calcium chloride dehydrate, 10.0 g manganese(II) chloride tetrahydrate, 1.0 g cobalt(II) chloride hexahydrate and 8.0 g iron(III) chloride. The flask is filled to 100 ml with distilled water and the contents dissolved. The solution is to be stored at 5-8° C.

Resazurin Solution

Under the safety of a fume-hood, wearing safety glasses and nitrile gloves, 100 mg resazurin is weighed into a 100 ml measuring Flask. The flask is filled to 100 ml with distilled water and the contents dissolved. Resazurin solution is to be stored at 5-8° C.

Reduction Solution

The reduction solution is prepared fresh prior to the fermentation in a 50 ml Falcon tube. The solution contains 385 mg sodium sulphide hydrate added to 22.5 ml distilled water and 2.7 ml sodium hydroxide 1 M. The solution is stirred until fully dissolved.

Rumen Fluid Collection

For the HFT-runs the rumen fluid from four sheep designated as animals for experimentation is used. The use of sheep in place of cattle for the rumen fluid samples has shown little to no difference in the fermentation as well as being easier to keep (Menke et al., 1979). The rumen fluid (400 ml) is collected from the animals for research purposes that have been fitted with a fistula granting direct access to the rumen.

The sheep are fed a uniform diet over a period of time to retain the consistency of the rumen liquor. The animals should be fed the same forage throughout the day over a longer period of time. This ensures a constant quality and stability of the rumen fluid. The diet should cover all their nutritional needs. Furthermore, a rumen fluid sample is always taken from two sheep per test. This helps to ensure a greater consistency in the microorganism activity. To create the most unbiased results, the sheep are used in rotation on a weekly basis. Sampling of the rumen fluid should be carried out before the morning meal.

2. In Vitro Fermentation in Rumen Fluid

Preparation of the Rumen Fluid Buffer

A buffer medium stabilizes the rumen fluid by simulating the characteristics of the animal's saliva. The buffer medium contains a mass element solution, a carbonate buffer and a trace element solution. Reduction solution and a colour indicator (resazurin) are also added. The contents are mixed while being gassed with carbon dioxide at 39° C. to ensure the proper rumen conditions. The rumen fluid is added once the indicator has become colourless in a ratio of 1:2 to the buffer medium.

The rumen fluid buffer mixture is prepared in a 2 L Woulff bottle. The bottle is placed on a warm plate (39° C.) with magnetic stirrer and the reagents are added in the following order:
640 mL distilled water
320 mL buffer solution
320 mL mass element solution
162 μL trace element solution
Reduction solution (freshly prepared) and
1710 μL Resazurin.

The rumen fluid buffer mixture is then immediately placed under the fume hood in the pre-heated water bath, the dispenser attached and the CO₂ tube inserted through a small bottle neck directly into the mixture. Carbon dioxide should be applied for at least an hour before adding the rumen fluid to ensure the absence of oxygen; the color of the mixture should change from blue to pink. The rumen fluid (delivered the morning of) is kept incubated until use and is then poured through a sieve. Under anoxic conditions 675 ml of the filtered rumen fluid is measured and added to the rumen fluid buffer mixture. The CO₂ tube should now be pulled to just above the mixture's surface. This rumen fluid mixture is allowed to mix for 15 minutes.

Samples for Control and Dry Matter Analysis

For control three 25 ml samples are taken pH measurement in falcon tubes, one with rumen fluid, one with rumen fluid mixture and one with rumen fluid mixture and 300 mg DM TMR-substrate. (A total mixed ration-substrate combines maize, proteins feeds, minerals, vitamins and feed additives formulated in a single feed mix.) The pH of the three control samples is taken once before and once after the filling of the syringes.

A petri-dish is weighed and the weight noted. The dish is filled about half way with the sieved rumen fluid and the weight noted. The petri-dish is then placed in a drying oven at 105° C. to dry overnight. The dish is to be weighed the next day and the dry matter percentile can be calculated. An average dry matter content of 2% of the rumen fluid is expected.

Filling the Syringes and Incubation

Once the rumen fluid buffer mixture has been prepared and thoroughly mixed, the syringes can be filled. The fermentation takes place in a 100 ml graduated glass syringe sealed with a petroleum jelly covered plunger. For each test series six syringes are used.

The Enzyme preparations for testing can then be added at the correct concentration and volume of 100,000 ppm per 100 μl of the dilution through the silicon tube attached to the glass syringes. For the addition of a single enzyme preparation, 200 μl is added and when a mixture of the two preparations is used 100 μl of each is added. Using the dispenser on the Woulff bottle, 25 mL of rumen fluid mixture is dispensed into each of the pre-warmed glass syringe and any bubbles present are expelled. The clamp is locked and the volume noted and the syringes are then placed back in the incubator. The incubation period runs for a total of 24 hours.

The syringes are incubated at 40° C. under rotation for 24 hours. A gas measurement is taken at each 0, 2, 4, 8 and 24 hours. The amount of gas produced after the 24-hour period is evidence of how well the substrate was digested.

HFT Based on Degradation Rate and Hybrid Type with Enzymes Supplement

This experiment is run using a selected three of the eight maize hybrids. The substrates used are MH5, MH6 and MH7. The substrate MH5 is a flint hybrid, MH7 a dent maize and MH6 is an unknown hybrid. For each substrate a series of the following enzyme preparations is performed: protease only (200000 ppm), amylase only (200000 ppm) and a protease-amylase mix (100000 ppm each). The substrates are run in series of six whereas the standards (substrate only) are run in series of four. The design layout can be seen in the following table 1.

TABLE 1

| | | | | | | | Substrate | | | Total Enzyme | | Total Enzyme |
|---|---|---|---|---|---|---|---|---|---|---|---|---|
| Series | Syringes (6 repetitions) | | | | | | Ref. | DM | Enzyme I | Preparation | Enzyme II | Preparation |
| 5a | 1 | 11 | 21 | 31 | 41 | 51 | MH5 | 300 mg | Amylase | 200000 ppm | | |
| 5b | 2 | 12 | 22 | 32 | 42 | 52 | | 300 mg | | | Protease | 200000 ppm |
| 5c | 3 | 13 | 23 | 33 | 43 | 53 | | 300 mg | Amylase | 100000 ppm | Protease | 100000 ppm |
| 6a | 4 | 14 | 24 | 34 | 44 | 54 | MH6 | 300 mg | Amylase | 200000 ppm | | |
| 6b | 5 | 15 | 25 | 35 | 45 | 55 | | 300 mg | | | Protease | 200000 ppm |
| 6c | 6 | 16 | 26 | 36 | 46 | 56 | | 300 mg | Amylase | 100000 ppm | Protease | 100000 ppm |
| 7a | 7 | 17 | 27 | 37 | 47 | 57 | MH7 | 300 mg | Amylase | 200000 ppm | | |
| 7b | 8 | 18 | 28 | 38 | 48 | 58 | | 300 mg | | | Protease | 200000 ppm |
| 7c | 9 | 19 | 29 | 39 | 49 | 59 | | 300 mg | Amylase | 100000 ppm | Protease | 100000 ppm |

| | | | | | | | |
|---|---|---|---|---|---|---|---|
| 5 | 10 | 20 | 30 | 40 | MH5 | 300 mg | Blank5 (Maize, no enzyme) |
| 6 | 50 | 60 | 61 | 62 | MH6 | 300 mg | Blank6 (Maize, no enzyme) |
| 7 | 63 | 64 | 65 | 66 | MH7 | 300 mg | Blank7 (Maize, no enzyme) |

The HFT with enzymes is run twice on two separate days to ensure minimal difference in the rumen fluid activity and reproducibility.

HFT Based on Total Prolamin with Enzyme Supplement

An additional experiment was run comparing three maize hybrids with different prolamin content. The maize hybrids MH1 (low), MH2 (middle) and MH08 (high) were used. For each substrate two enzyme preparations and a mix of the two were tested. The design layout can be seen in the following table 2.

TABLE 2

| | | | | | | | Substrate | | | Total Enzyme | | Total Enzyme |
|---|---|---|---|---|---|---|---|---|---|---|---|---|
| Series | Syringes (6 repetitions) | | | | | | Ref. | DM | Enzyme I | Preparation | Enzyme II | Preparation |
| 8a | 1 | 11 | 21 | 31 | 41 | 51 | MH8 | 300 mg | Amylase | 200000 ppm | | |
| 8b | 2 | 12 | 22 | 32 | 42 | 52 | | 300 mg | | | Protease | 200000 ppm |
| 8c | 3 | 13 | 23 | 33 | 43 | 53 | | 300 mg | Amylase | 100000 ppm | Protease | 100000 ppm |
| 2a | 4 | 14 | 24 | 34 | 44 | 54 | MH2 | 300 mg | Amylase | 200000 ppm | | |
| 2b | 5 | 15 | 25 | 35 | 45 | 55 | | 300 mg | | | Protease | 200000 ppm |
| 2c | 6 | 16 | 26 | 36 | 46 | 56 | | 300 mg | Amylase | 100000 ppm | Protease | 100000 ppm |
| 1a | 7 | 17 | 27 | 37 | 47 | 57 | MH1 | 300 mg | Amylase | 200000 ppm | | |
| 1b | 8 | 18 | 28 | 38 | 48 | 58 | | 300 mg | | | Protease | 200000 ppm |
| 1c | 9 | 19 | 29 | 39 | 49 | 59 | | 300 mg | Amylase | 100000 ppm | Protease | 100000 ppm |

| | | | | | | | |
|---|---|---|---|---|---|---|---|
| 8 | 10 | 20 | 30 | 40 | MH8 | 300 mg | Blank8 (Maize, no enzyme) |
| 2 | 50 | 60 | 61 | 62 | MH2 | 300 mg | Blank2 (Maize, no enzyme) |
| 1 | 63 | 64 | 65 | 66 | MH1 | 300 mg | Blank1 (Maize, no enzyme) |

The HFT based on prolamin content is run twice on two separate days to ensure minimal difference in the rumen fluid activity and reproducibility.

3. Results

The supplementation of a specific protease on maize kernel meal in the HFT system showed no significant advantage over the supplementation of a specific amylase. The addition of an amylase-protease mixture where the concentration of each enzyme is only half of that which is used in the tests with a single enzyme preparation shows significant results. The attempt to find a correlation between the amount of crude protein content of maize and the effects of a protease treatment alone or in the enzyme mix has been inconclusive. There was also no consistent correlation between the effects of a protease treatment, alone or in combination, with an alpha-amylase on the total prolamin content of maize.

In this experiment the addition of a specific protease preparation or an alpha-amylase preparation to the ruminant diet displayed significant effects on the rate of coarse maize kernel meal digestion. The effects on the rate of gas production of each enzyme treatment alone at a concentration of 200,000 ppm do not appear to differ. The effects are greatest between two and four hours incubation time.

The addition of an enzyme mix containing 100,000 ppm of each an alpha-amylase and a protease on a variety of maize hybrids exhibited a clear synergistic effect. Although only half the amount of a single enzyme is in the mix as compared to the single enzyme treatments the rate of gas production is higher. The supplementation of the ruminant diet with an amylase preparation is already marketed and shown to positively affect the rate of digestion of starchy feedstuffs. The addition of a protease appears to release more starch boosting the effects of an amylase in the rumen.

### Example 3

### Maize-Degradation In Vivo

In this present study eight non-lactating Holstein-Friesian dairy cows (live body weight about 650 kg) were used to measure the in situ rumen degradation characteristics. The cows were provided with rumen cannulas (Bar Diamond Inc., Parma, Id., USA) with 10 cm internal width. During the in situ experiment, the cows were individually penned in a clean and air conditioned stable (temperature 20° C.). Clean fresh water and salts blocks were offered for free choice. The ration which was used during the experimental period is presented in Table 3. Daily dry matter intake was 7.0 kg and the cows were fed twice daily in two equal portions at 07.00 a.m. and at 04.00 p.m.

The tested enzyme products were RONOZYME RumiStar 600 (CT) and RONOZYME ProAct (CT). The different treatments were: Control (Con, no enzyme addition), addition of amylase (Amy) and addition of protease (Prot) and Amy.

The enzymes were added individually every meal to the enzyme treated cows (amylase: 0.5 g/kg dry matter (DM); combination of both enzymes: 0.25 g amylase/kg DM+0.1 g protease/kg DM) and thoroughly mixed to the feed ingredients. The total mixed ration was fed for 21 days before start of the experimental phase (adaptation phase) and extended throughout the experimental period (experimental phase). After each experimental phase there was a wash-out phase for 8 days without any enzyme addition.

Fistulated cows were used as 8×4 Latin Square with factorial arrangement of treatment in four periods. During each period 2 cows received the control treatment, 2 cows received the amylase treatment and 2 cows received the combination of both enzymes. In the following 3 periods the treatments changed, so that each cow received every treatment.

TABLE 3
Composition of the experimental diet

| Feedstuff | Percentage of the TMR | DM content (%) |
|---|---|---|
| Maize silage | 49 | 33.30 |
| Grass silage | 15 | 38.34 |
| Grain maize | 20 | 89.10 |
| Soybean meal | 6 | 88.78 |
| Hay | 10 | 92.71 |

Experimental Design

In this study the in situ dry matter disappearance (%) of the TMR (total mixed rations) and its individual components was studied by using the nylon bag technique (Ørskov & McDonald, J. Agric. Sci. (Camb.) 92, 1979, 499-503).

In contrast to common in situ studies e.g. on protein degradability of distinct feed components added to the ration, this example focused also on the impact of the enzyme addition on rumen fermentation kinetics of the entire ration.

Another aspect was preparation of feed samples. Usually, the feed samples are dried and ground but this might modulate fermentation kinetics inside the nylon bag compared to the situation outside the bag. Therefore, the feed sample preparation was done with fresh materials.

The bags (10×20 cm) used in this study had a pore size of 50 Hm (R1020, Dohod Technology, Fairport, USA). Four grams of DM were weighed into previously labelled, dried (at 60° C. for 48 h) and weighed bags, which were incubated in the rumen of the fistulated cows. For TMR bags the individual components were weighed and placed into the bags in the same proportion as present in the ration.

In order to guarantee homogeneous presence of the enzyme products in all tested material, the enzymes were admixed to each of the nylon bags in the same proportion as it was presented in the respective TMR. Nylon bags of the control treatment received no addition of enzymes.

Twenty-four bags were prepared for each cow at each incubation time (4 bags from each component of the TMR and the TMR itself).

Additionally, 24 “0-hour” nylon bags were prepared (1 bag for each treatment and feedstuff) to serve as control at each incubation time.

The nylon bags were incubated in the rumen of the eight cows just before the morning feeding at 07.00 a.m. The different incubation times were: 1, 2, 3, 4, 5, 6, 9, 12, 24, and 48 hours.

Bags were removed from the rumen (all in-all out system) and were immediately put into ice water to stop microbial activity. After that the bags were put together with the corresponding 0-hour bags into a washing tank and washed with tap water until the water runs clear. Thereafter, the bags were washed in a washing machine with cold water without a spin cycle for 19 minutes. Afterwards the bags were freeze dried and weighed again to determine the in situ dry matter disappearance.

Ruminal fluid samples (about 200 ml) were collected from each animal at the short term incubation periods (1, 2, 3, 4, 5, 6, and 9 h) at the onset of incubation and at removal of nylon bags. Samples were divided into two portions. One portion was used to measure rumen pH directly and then was centrifuged and frozen to be used for measure the ammonia nitrogen. The second part was centrifuged and 10 ml of the supernatant was preserved and frozen to determine volatile fatty acids later on.

The in situ dry matter disappearance (ISDMD) of incubated material at a certain incubation time was calculated as percentage of dry matter loss before and after incubation:

$$ISDMD ( \% ) = \frac{\begin{matrix}Weight before incubation ( g ) - \\ Weight after incubation ( g )\end{matrix}}{Weight before incubation ( g )} \times 100$$

ISDMD from bags within cows, treatment and incubation times as well as corresponding rumen fluid pH values were subjected to 2-way analysis of variance with GLM procedures of SAS Vers. 9.4 (SAS Institute Inc., Cary, USA).

Differences between treatments were assessed for statistical significance by Duncan-Test (p<0.05).

Results

In situ dry matter disappearance (ISDMD) of the different feed ingredients with the three different treatments is presented in Table 4-9. SEM represents the standard error of means, different superscripts show significant differences between treatments.

The ISDMD of the TMR (Table 4) showed differences between the treatments in the short incubation times. The combination of amylase and protease showed a higher ISDMD from 1 h up to 5 h compared to the control. The same effect could be observed at 9 h. After this point in time there was no consistent effect of treatments on ISDMD.

The ISDMD of grass silage (Table 5) showed no quantitatively relevant effect of enzyme addition.

The ISDMD of maize silage (Table 6) indicated differences between the treatments. The combination of both enzymes showed a higher ISDMD from 1 h up to 24 h compared to the control.

The ISDMD of maize grain (Table 7) also indicated differences between treatments. The combination of amylase and protease showed a higher ISDMD from 1 h up to 24 h compared to the control.

The ISDMD of soybean meal (Table 8) and hay (Table 9) showed no quantitatively relevant effect of enzyme addition.

TABLE 4
In situ dry matter disappearance (%) of the TMR
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 19.84 | 26.10 | 25.91 | 27.88 | 28.50 | 30.46 | 32.45 | 36.90 | 43.24 | 60.62 | 76.97 |
| Amy | 21.44 | 27.75 | 28.16 | 28.83 | 30.82 | 32.64 | 33.13 | 38.17 | 40.38 | 62.69 | 76.86 |
| Amy + Prot | 22.15 | 28.37 | 28.27 | 30.43 | 31.64 | 32.58 | 33.94 | 39.48 | 44.51 | 63.67 | 77.48 |
| SEM | — | 1.00 | 1.01 | 0.97 | 1.09 | 0.90 | 1.18 | 1.30 | 1.59 | 1.64 | 1.40 |
| p-value | — | <0.01 | <0.01 | <0.01 | <0.01 | <0.01 | <0.1 | <0.01 | <0.01 | <0.05 | <0.05 |

TABLE 5
In situ dry matter disappearance (%) of grass silage
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 29.10 | 31.82 | 33.71 | 34.64 | 35.34 | 36.35 | 38.59 | 43.65 | 49.31 | 67.97 | 81.07 |
| Amy | 28.25 | 31.01 | 32.01 | 32.88 | 34.04 | 35.44 | 37.14 | 41.48 | 46.00 | 67.44 | 79.27 |
| Amy + Prot | 28.18 | 31.86 | 32.75 | 34.02 | 34.89 | 36.67 | 37.19 | 43.83 | 48.96 | 66.92 | 79.70 |
| SEM | — | 0.32 | 0.84 | 0.64 | 0.65 | 0.55 | 0.98 | 1.22 | 1.85 | 1.85 | 1.09 |
| p-value | — | <0.01 | <0.05 | <0.01 | <0.05 | <0.01 | <0.1 | <0.05 | <0.05 | n.s. | <0.05 |

TABLE 6
In situ dry matter disappearance (%) of maize silage
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 21.68 | 27.88 | 27.86 | 29.83 | 30.36 | 32.73 | 34.59 | 37.77 | 42.8 | 59.40 | 75.58 |
| Amy | 24.77 | 28.92 | 28.76 | 32.11 | 33.30 | 35.33 | 35.45 | 40.23 | 43.91 | 60.14 | 75.90 |
| Amy + Prot | 26.95 | 32.46 | 31.82 | 33.87 | 33.27 | 37.17 | 35.65 | 41.67 | 47.66 | 62.47 | 75.81 |
| SEM | — | 1.42 | 1.36 | 1.19 | 1.38 | 1.69 | 1.84 | 1.66 | 1.72 | 1.68 | 1.54 |
| p-value | — | <0.01 | <0.01 | <0.01 | <0.01 | <0.01 | <0.05 | <0.01 | <0.01 | <0.05 | n.s. |

TABLE 7
In situ dry matter disappearance (%) of grain maize
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 22.05 | 28.15 | 30.17 | 31.92 | 33.30 | 35.18 | 36.81 | 41.31 | 46.34 | 67.40 | 94.48 |
| Amy | 20.77 | 27.74 | 29.92 | 31.60 | 32.71 | 34.89 | 35.95 | 39.82 | 45.10 | 68.97 | 91.96 |
| Amy + Prot | 23.18 | 30.17 | 32.25 | 35.09 | 35.06 | 37.21 | 38.80 | 43.84 | 49.80 | 71.17 | 93.60 |
| SEM | — | 0.59 | 0.41 | 0.69 | 0.77 | 0.65 | 0.73 | 0.77 | 1.21 | 1.96 | 1.31 |
| p-value | — | <0.01 | <0.01 | <0.01 | <0.01 | <0.01 | <0.01 | <0.01 | <0.01 | <0.01 | <0.05 |

TABLE 8
In situ dry matter disappearance (%) of soybean meal
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 26.67 | 30.90 | 33.84 | 36.12 | 39.18 | 42.03 | 45.02 | 59.40 | 68.95 | 93.30 | 98.05 |
| Amy | 26.30 | 31.00 | 33.16 | 35.61 | 39.11 | 42.16 | 46.08 | 56.91 | 64.73 | 91.63 | 97.94 |
| Amy + Prot | 25.64 | 31.00 | 33.20 | 37.04 | 39.01 | 42.79 | 46.33 | 61.78 | 71.22 | 92.42 | 98.05 |
| SEM | — | 0.46 | 0.48 | 0.78 | 0.91 | 1.27 | 1.65 | 2.55 | 3.36 | 1.59 | 0.28 |
| p-value | — | n.s. | <0.1 | <0.01 | n.s. | n.s. | n.s. | <0.01 | <0.05 | n.s. | n.s. |

TABLE 9
In situ dry matter disappearance (%) of hay
Incubation time (h)

| Treatment | 0 | 1 | 2 | 3 | 4 | 5 | 6 | 9 | 12 | 24 | 48 |
|---|---|---|---|---|---|---|---|---|---|---|---|
| Con | 10.57 | 13.47 | 14.96 | 15.89 | 16.64 | 17.60 | 18.45 | 20.91 | 22.95 | 35.99 | 51.06 |
| Amy | 9.96 | 14.17 | 15.07 | 15.77 | 16.60 | 17.65 | 18.71 | 21.02 | 22.75 | 36.04 | 50.07 |
| Amy + Prot | 10.18 | 13.63 | 15.01 | 15.58 | 16.75 | 17.15 | 18.22 | 20.32 | 23.14 | 36.4 | 49.55 |
| SEM | — | 0.35 | 0.27 | 0.29 | 0.26 | 0.31 | 0.35 | 0.45 | 0.72 | 1.19 | 1.01 |
| p-value | — | <0.05 | n.s. | n.s. | n.s. | <0.1 | n.s. | n.s. | n.s. | <0.05 | <0.1 |

The results regarding the in situ dry matter disappearance show that the addition of the tested enzymes accelerates the ruminal degradation of maize. The addition of the combination of both enzymes leads to a consistently higher ISDMD in maize grain from 1 h up to 24 h of incubation. The ISDMD of maize silage was also affected by the enzyme treatments. There, a higher ISDMD could be observed for the amylase+protease treatment from 1 h up to 24 h of incubation. The degradation characteristics of the TMR were similar to maize silage, due to the high percentage (49%) of maize silage in the TMR.

1-11. (canceled)

12. A method for improving the digestibility of maize and/or maize silages in feed for animals of the subfamily Bovinae, comprising treating a maize source with an effective amount of one or more proteolytic enzymes in combination with at least one amylase.

13. A method for increasing the milk yield, increasing the weight gain or improving the feed conversion ratio of an animal of the subfamily Bovinae, comprising adding at least one bacterial amylase and one or more proteolytic enzymes to the feed which comprises maize and/or maize silages.

14. A feed composition comprising at least one bacterial amylase, one or more proteolytic enzymes, and maize and/or maize silage.
